(19) United States
(12) Patent Application Publication
(10) Pub. No.: US 2024/0090852 A1
(43) Pub. Date: Mar. 21, 2024

(54) ANALYTE MONITORING SYSTEMS AND METHODS

(71) Applicant: ABBOTT DIABETES CARE INC., Alameda, CA (US)

(72) Inventors: Rosina Bosco, Boulder, CO (US); Felipe Escobar Pinedo, Atlantico (CO); Bjorn Lorenzo-Van Der Meer, Emeryville, CA (US); Jessica Rose Floeh, Baltimore, MD (US); Kimberly Hilton, San Francisco, CA (US); Eesha M. Ambike, San Jose, CA (US)

(21) Appl. No.: 18/369,100

(22) Filed: Sep. 15, 2023

Related U.S. Application Data
(60) Provisional application No. 63/538,592, filed on Sep. 15, 2023.
(60) Provisional application No. 63/407,430, filed on Sep. 16, 2022.

(51) Int. Cl. A61B 5/00; A61B 5/145

(52) U.S. Cl. CPC A61B 5/743; A61B 5/0004; A61B 5/14532; A61B 5/7275; A61B 5/7435; A61B 5/746

(57) ABSTRACT

Improved graphical user interfaces for analyte monitoring systems are provided. For example, disclosed herein are various embodiments of GUIs, each of which include a graph portion comprising a trend line indicative of a user's analyte levels over a predetermined time period, and a plurality of summary metrics comprising a plurality of minimum analyte levels and maximum analyte levels associated with a plurality of time increments within the predetermined time period. In many embodiments, the plurality of minimum analyte levels and maximum analyte levels are aligned with an x-axis of the graph portion.

### CROSS-REFERENCE TO RELATED APPLICATIONS

This application claims priority to U.S. Provisional Application No. 63/538,592, filed Sep. 15, 2023, and U.S. Provisional Application No. 63/407,430, filed Sep. 16, 2022, both of which are hereby expressly incorporated by reference in their entireties for all purposes.

### FIELD

The subject matter described herein relates generally to improvements to analyte monitoring systems, as well as computer-related methods and devices relating thereto.

### BACKGROUND

The detection and/or monitoring of analyte levels, such as glucose, ketones, lactate, oxygen, hemoglobin A1C, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with a bodily fluid. The sensor control device may also be configured to transmit analyte data to another device, from which the individual, her health care provider (“HCP”), or a caregiver can review the data and make therapy decisions.

Despite their advantages, however, some people are reluctant to use analyte monitoring systems for various reasons, including the complexity and volume of data presented, a learning curve associated with the software and user interfaces for analyte monitoring systems, and an overall paucity of actionable information presented.

Thus, needs exist for improved reporting interfaces and software for analyte monitoring systems, as well as methods and devices relating thereto, that are robust, user-friendly, and provide for timely and actionable responses.

### SUMMARY

Provided herein are example embodiments of improvements to in vivo analyte monitoring systems, and computer-related methods and devices relating thereto. According to some embodiments, a graphical user interface (“GUI”) of an analyte monitoring system is provided, wherein the GUI comprises a graph portion comprising a trend line indicative of a user's analyte levels over a predetermined time period, wherein the trend line is based on data indicative of the user's analyte levels received from a sensor control device. In some embodiments, the graph portion comprises a graphical display of the user's analyte levels over the predetermined time period. The graph portion may have an x-axis representing the predetermined time period. For example, the graph portion may comprise a plurality of data points representing the user's analyte levels over the predetermined time period (for example, in the form of a scatter graph). In some examples, the plurality of data points may be joined together by a line (for example, in the form a line graph). In other examples, the plurality of data points may be discrete and not joined together by a line. According to an aspect of some embodiments, the GUI further comprises a plurality of summary metrics associated with the predetermined time period. In some examples, the summary metrics are associated with the user's analyte levels. According to an aspect of some embodiments, the plurality of summary metrics includes a plurality of minimum analyte levels and maximum analyte levels associated with a plurality of time increments within the predetermined time period. In other words, the plurality of summary metrics may include a minimum analyte level and a maximum analyte level for each time increment of a plurality of time increments within the predetermined time period. In some embodiments, the predetermined time period is one day. For example, the plurality of time increments may comprise twenty-four (24) one-hour increments. In other words, each time increment may have a duration of one hour, for example. In some embodiments, the plurality of minimum analyte levels and maximum analyte levels are aligned with an x-axis of the graph portion. For example, each minimum and maximum analyte level may be aligned with a corresponding time interval on the x-axis, and the plurality of minimum and maximum analyte levels are displayed along the x-axis for the different time intervals.

Many of the embodiments provided herein are improved report GUIs or GUI features for analyte monitoring systems that are highly intuitive, user-friendly, and provide for rapid access to physiological information of a user. More specifically, these embodiments allow a user to easily navigate through and between different user interfaces that can quickly indicate to the user various physiological conditions and/or actionable responses, without requiring the user (or an HCP) to go through the arduous task of examining large volumes of analyte data.

The improvements to the GUIs in the various aspects described and claimed herein produce a technical effect at least in that they assist the user of the device to operate the device more accurately, more efficiently, and more safely. It will be appreciated that the information that is provided to the user via the report GUI, the order in which that information is provided, and the clarity with which that information is structured can have a significant effect on the way the user interacts with the system and the way the system operates. The report GUI therefore guides the user in the technical task of operating the system to take the necessary readings and/or obtain information accurately and efficiently. Other improvements and advantages are provided as well. The various configurations of these devices are described in detail by way of the embodiments which are only examples.

Other systems, devices, methods, features and advantages of the subject matter described herein will be or will become apparent to one with skill in the art upon examination of the following figures and detailed description. It is intended that all such additional systems, devices, methods, features, and advantages be included within this description, be within the scope of the subject matter described herein, and be protected by the accompanying claims. Aspects of the embodiments are set out in the independent claims and preferred features are set out in the dependent claims. The preferred features of the dependent claims may be provided in combination in a single embodiment and preferred features of one aspect may be provided in conjunction with other aspects. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of an analyte monitoring system comprising a sensor applicator, a sensor control device, a reader device, a network, a trusted computer system, and a local computer system.

FIG. 2A is a block diagram depicting an example embodiment of a reader device.

FIGS. 2B and 2C are block diagrams depicting example embodiments of sensor control devices.

FIGS. 3A to 3L are example embodiments of various graphical user interfaces (“GUIs”) and reporting GUIs, each of which is indicative of a user's analyte levels and a plurality of summary metrics during a predetermined time period.

FIG. 4A is an example embodiment of a report settings GUI for enabling and/or disabling the display of minimum analyte levels and maximum analyte levels.

FIGS. 4B to 4E are example embodiments of GUIs and reporting GUIs, where the display of minimum analyte levels and maximum analyte levels have been enabled or disabled.

FIG. 5 is an example embodiment of a reporting GUI indicative of analyte levels and summary metrics for multiple analytes during a plurality of predetermined time periods.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include GUIs, software, and digital interfaces for analyte monitoring systems, and methods and devices relating thereto. Accordingly, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices, reader devices, local computer systems, and trusted computer systems are disclosed, and these devices and systems can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps.

Improved reporting graphical user interfaces for analyte monitoring systems are provided. For example, disclosed herein are various embodiments of graphical user interfaces (“GUIs”). The GUIs are highly intuitive, user-friendly, and provide for rapid access to physiological information of a user. In sum, these embodiments provide for a robust, user-friendly interfaces that can increase user engagement with the analyte monitoring system and provide for timely and actionable responses by the user, to name a few advantages. Other improvements and advantages are provided as well. The various configurations of these devices are described in detail by way of the embodiments which are only examples.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

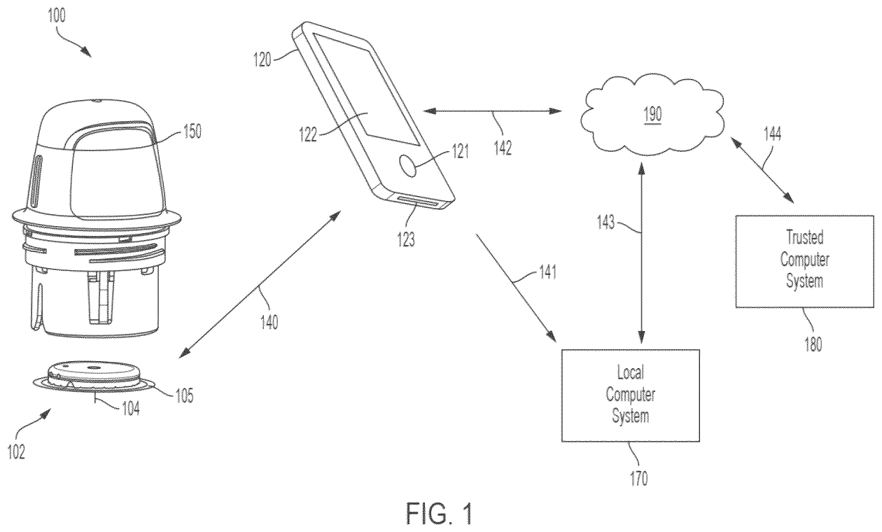

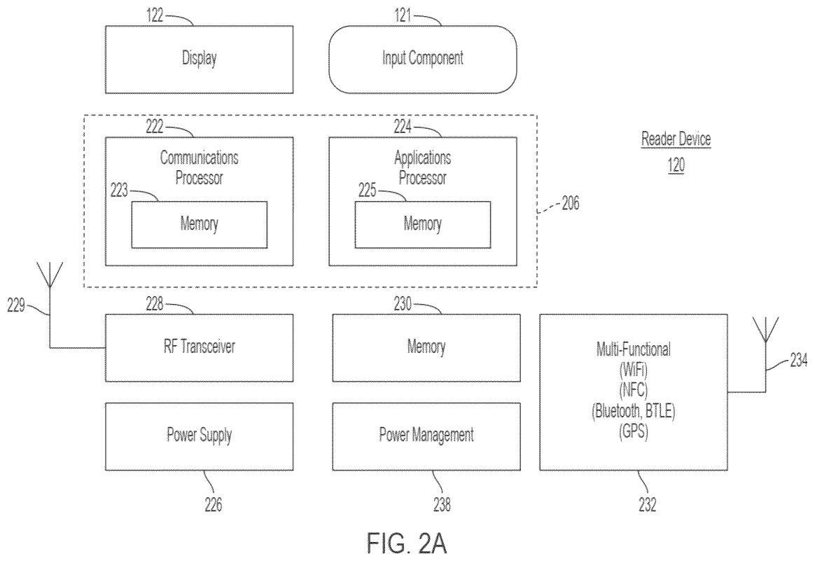

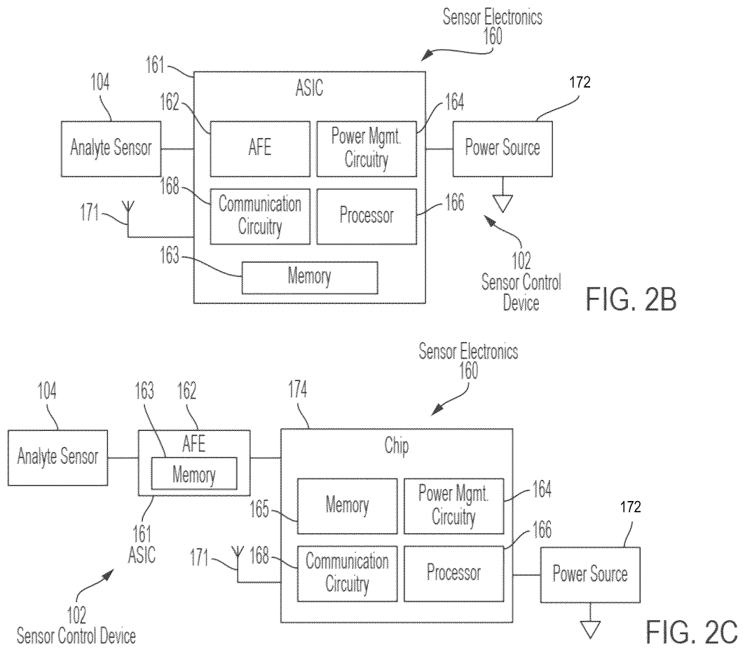

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 120 using screen 122 (which, in many embodiments, can comprise a touchscreen), and input 121 (which may be part of a touchscreen). A device battery of reader device 120 can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a cloud-based platform or server, and can provide for authentication services, secured data storage, report generation, and can communicate via communications path 144 with network 190 by wired or wireless technique. In addition, although FIG. 1 depicts trusted computer system 180 and local computer system 170 communicating with a single sensor control device 102 and a single reader device 120, it will be appreciated by those of skill in the art that local computer system 170 and/or trusted computer system 180 are each capable of being in wired or wireless communication with a plurality of reader devices and sensor control devices.

### Example Embodiment of Reader Device

FIG. 2A is a block diagram depicting an example embodiment of a reader device 120, which, in some embodiments, can comprise a smart phone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232, which can comprise wireless communication circuitry, and which can be configured to communicate over Wi-Fi, NFC, Bluetooth, BTLE, and GPS with an antenna 234. As understood by one of skill in the art, these components are electrically and communicatively coupled in a manner to make a functional device. The reader device 120 may be provided in combination with the sensor control device 102 to form an analyte monitoring system, or may be provided in isolation (for example, for use with a sensor control device 102). The reader device 120 and/or the sensor control device 102 may be provided in combination with a sensor applicator 150 for delivering the sensor control device 102 to the user's skin. For example, the sensor applicator 150 may be used to insert a portion of the analyte sensor 104 into the user's skin.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. The analyte sensor 104 may comprise a portion configured to be inserted into the user, for example to position a portion in contact with bodily fluid (e.g., interstitial fluid). In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete data and trend values, etc. This data can then be provided to communication circuitry 168 for sending, by way of antenna 171, to reader device 120 (not shown), for example, where minimal further processing is needed by the resident software application to display the data. According to some embodiments, for example, a current glucose value can be transmitted from sensor control device 102 to reader device 120 periodically (for example, every minute). In some examples, historical glucose values can be transmitted from sensor control device 102 to reader device 120 periodically (for example, every five minutes).

In some embodiments, to conserve power and processing resources on sensor control device 102, digital data received from AFE 162 can be sent to reader device 120 (not shown) with minimal or no processing. In still other embodiments, processor 166 can be configured to generate certain predetermined data types (e.g., current glucose value, historical glucose values) either for storage in memory 163 or transmission to reader device 120 (not shown), and to ascertain certain alarm conditions (e.g., sensor fault conditions), while other processing and alarm functions (e.g., high/low glucose threshold alarms) can be performed on reader device 120. Those of skill in the art will understand that the methods, functions, and interfaces described herein can be performed—in whole or in part—by processing circuitry on sensor control device 102, reader device 120, local computer system 170, or trusted computer system 180.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 may include memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Graphical User Interfaces for Analyte Monitoring Systems

Described herein are example embodiments of GUIs for analyte monitoring systems. As an initial matter, it will be understood by those of skill in the art that the GUIs described herein comprise instructions stored in a memory of trusted computer system 180, reader device 120, local computer system 170, and/or any other device or system that is part of, or in communication with, analyte monitoring system 100. These instructions, when executed by one or more processors of the trusted computer system 180, reader device 120, local computer system 170, or other device or system of analyte monitoring system 100, cause the one or more processors to perform the method steps and/or output the GUIs described herein. Those of skill in the art will further recognize that the GUIs described herein can be stored as instructions in the memory of a single centralized device or, in the alternative, can be distributed across multiple discrete devices in geographically dispersed locations.

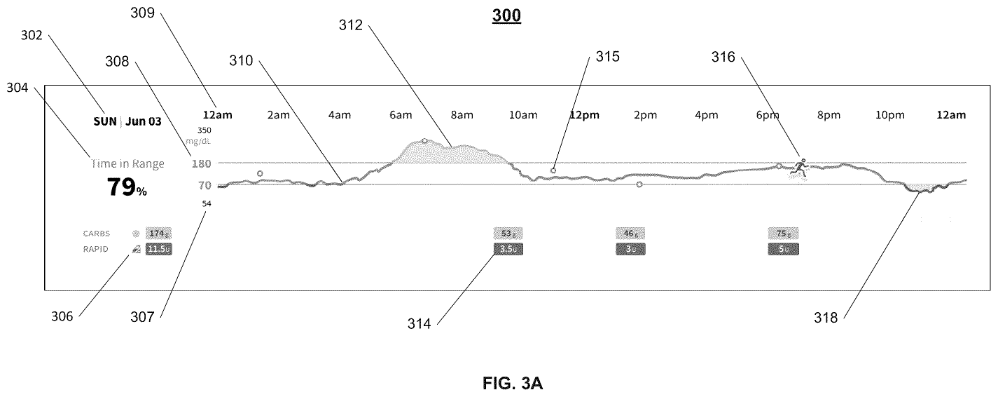

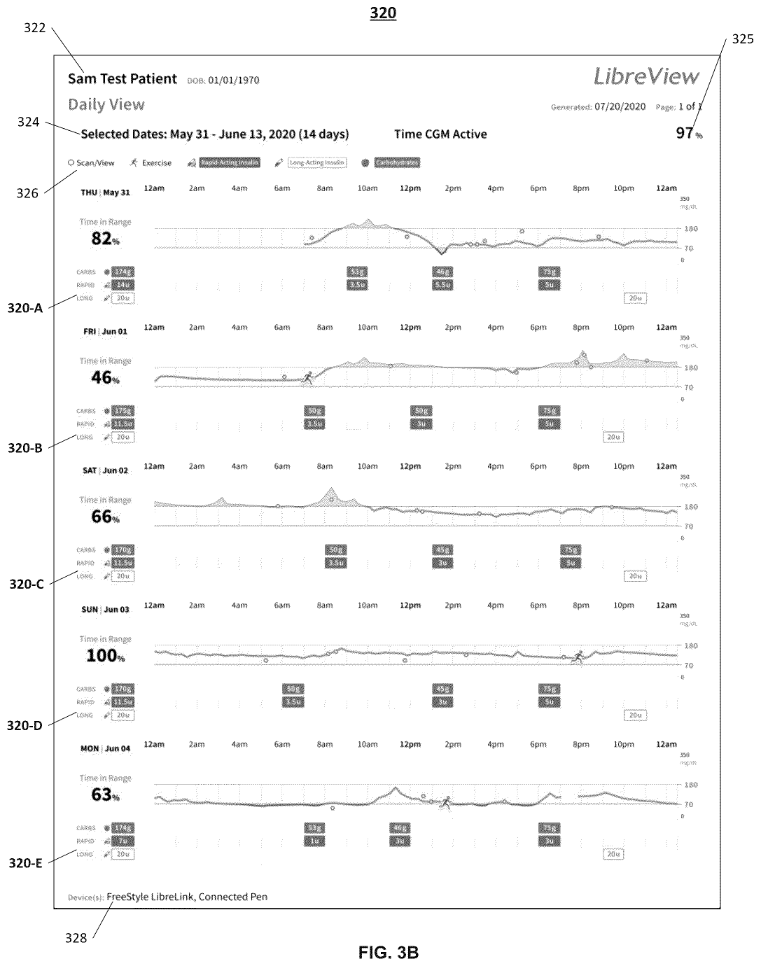

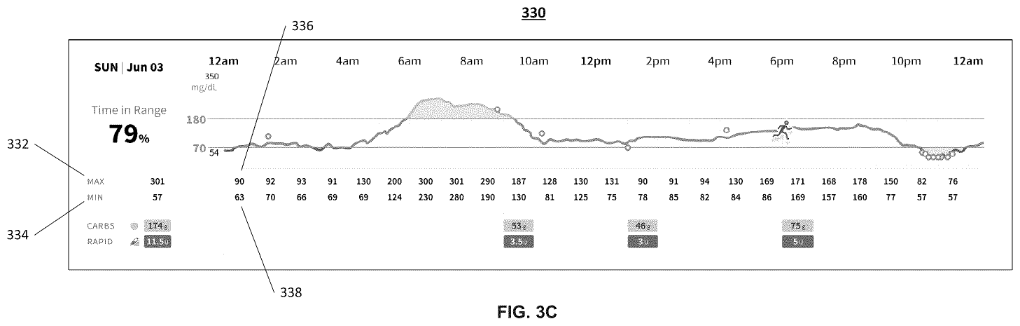

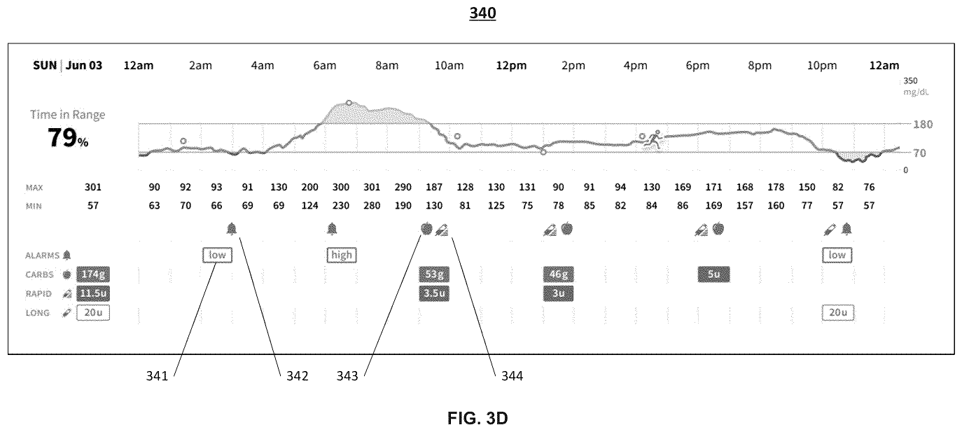

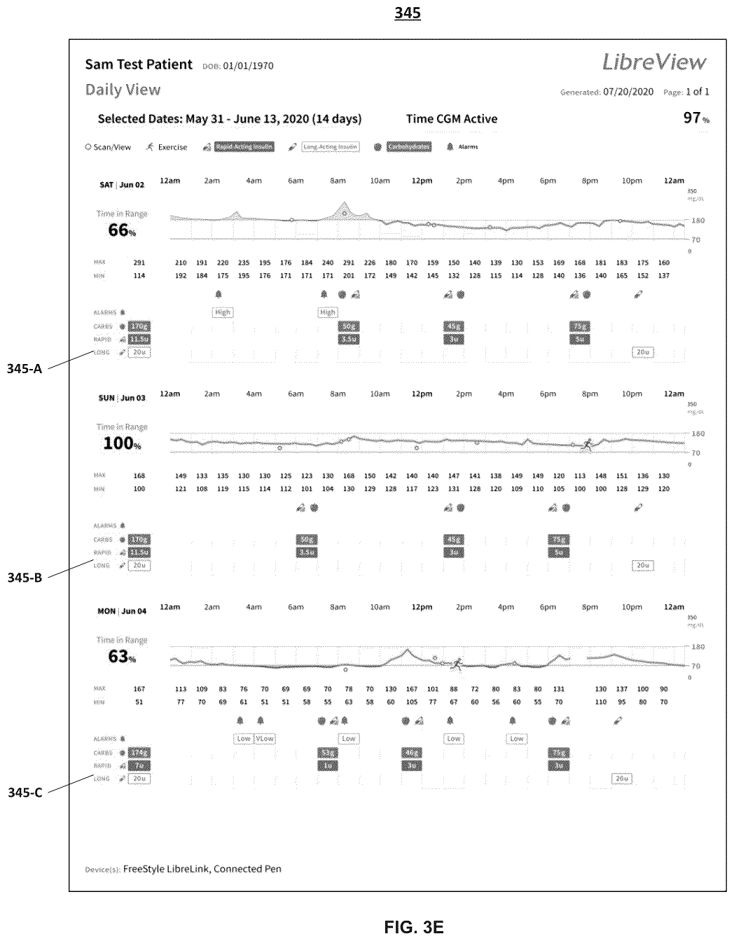

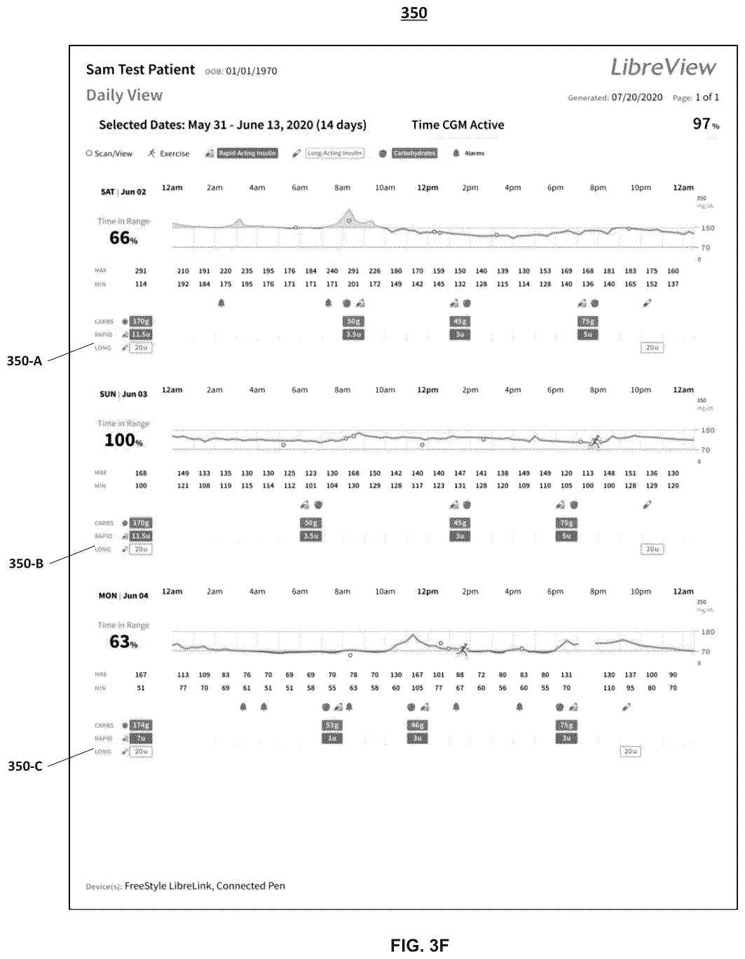

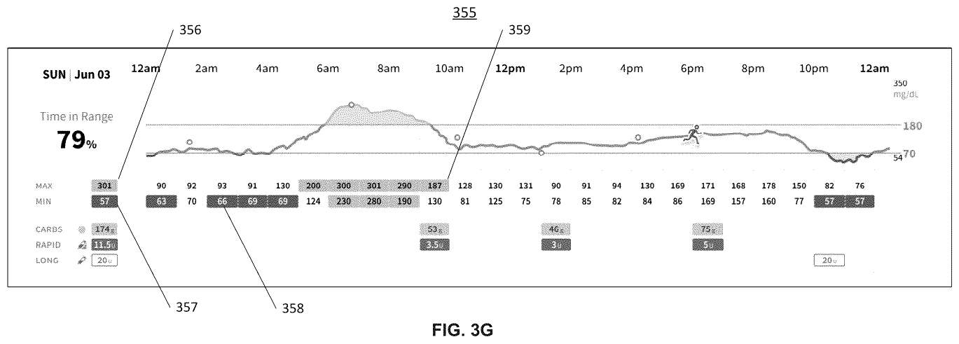

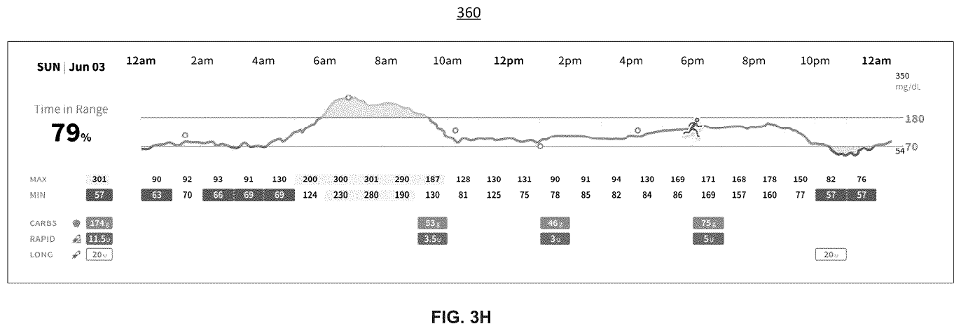

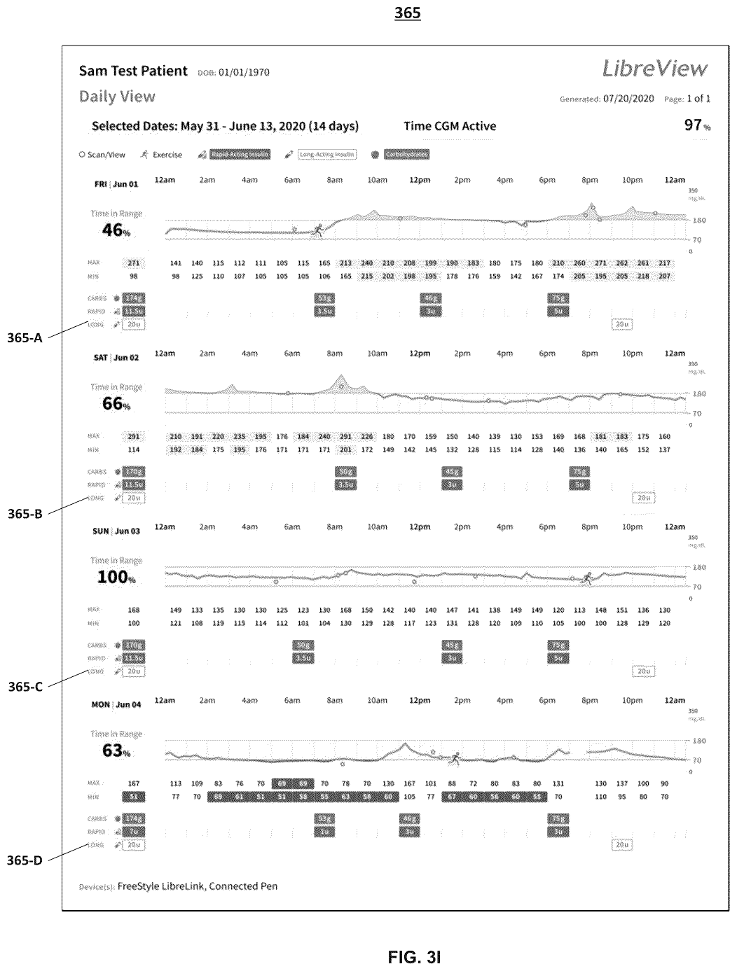

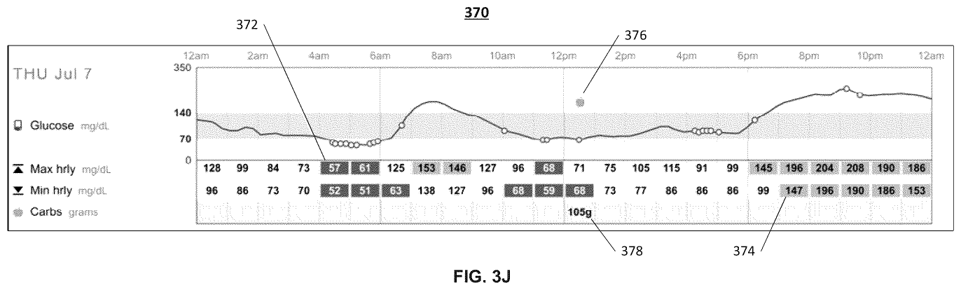

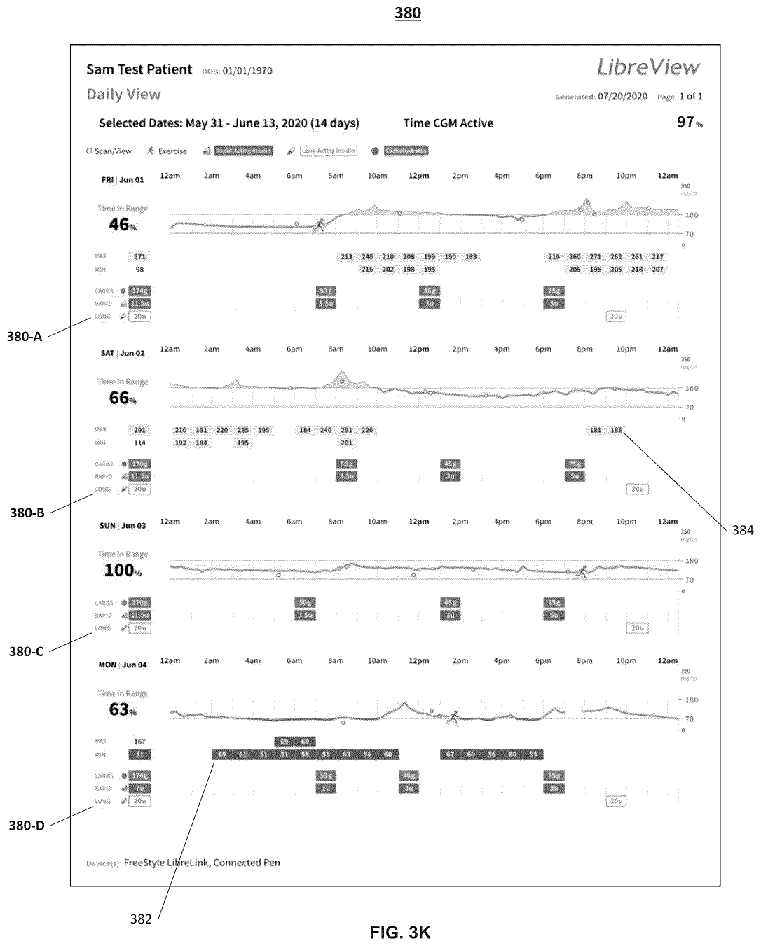

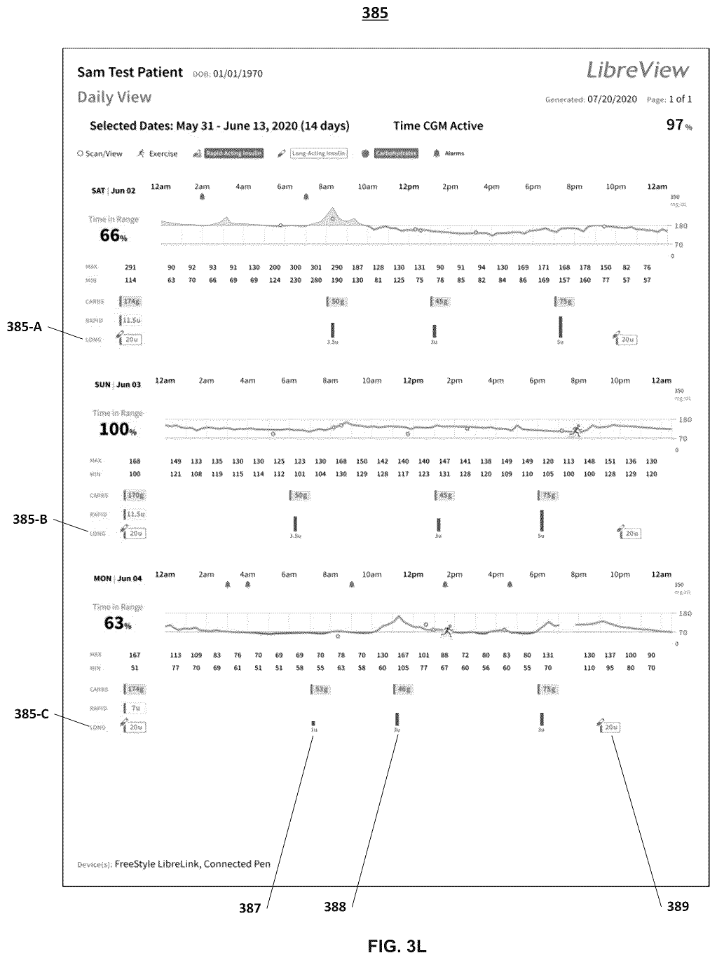

FIG. 3A depicts an example embodiment of a GUI 300 for an analyte monitoring system, in which GUI 300 depicts a user's analyte levels over a predetermined time period in conjunction with a plurality of summary metrics. Generally, GUI 300 can display, in a single user-friendly format, a graph representing the user's analyte levels over the predetermined time period, accompanied by a plurality of useful summary metrics based on, or relating to, analyte data obtained from an analyte monitoring system. Further, the plurality of summary metrics displayed in GUI 300 can be associated with the predetermined time period, e.g., a day, a week, or a month. In other words, the plurality of summary metrics may relate to analyte data from the predetermined time period. For example, as shown in FIG. 3A, GUI 300 is configured to display a graph of the user's analyte levels and a plurality of summary metrics for one day, as indicated by date 302 (e.g., “SUN, Jun 03”).

According to one aspect of the embodiments, GUI 300 can include a graph portion comprising an x-axis 309 based on units of time, and a y-axis 307 indicative of an analyte concentration. For example, GUI 300 includes an x-axis 309, labeled in two-hour increments across a twenty-four hour period, and a y-axis 307, labeled in units of milligrams of glucose per deciliter (mg/dL). Those of skill in the art will appreciate that the x-axis and/or the y-axis can be labeled with other increments or units. For example, the x-axis can be labeled in thirty-minute increments, one-hour increments, or four-hour increments, etc. As another example, the y-axis can be displayed in units of millimoles of glucose per liter (mmol/L).

According to another aspect of the embodiments, the graph portion of GUI 300 can further display a high analyte threshold value (“180 mg/dL”) and a low analyte threshold value (“70 mg/dL”) for a target analyte range 308, as indicated by the numeric labels on the left side. In some embodiments, the numeric values for target analyte range 308 can be a different color from the labels on the x-axis and/or y-axis, so as to make them more distinctive. Furthermore, in some embodiments, a plurality of lines, each of which reflects a corresponding threshold value of the target analyte range 308, can extend across the graph portion to provide a visual reference with respect to the relationship at any point-in-time between the user's analyte levels and target analyte range 308. For example, the lines may include a high analyte threshold line and a low analyte threshold line. In some examples, the lines may be colored, for example with the lines for each threshold being in a different color. This allows the user to more easily compare their analyte levels at particular points-in-time to the target analyte range 308, thereby improving patient safety. In addition, according to some embodiments, the target analyte range 308 can be configured by the user, for example, by adjusting either or both of the high analyte threshold value or the low analyte threshold value. In some embodiments, the lines for the threshold values may be provided without the numeric labels.

Referring still to FIG. 3A, graph portion of GUI 300 can include a trend line 310 showing the user's analyte concentrations over the predetermined time period. According to one aspect of the embodiments, certain portions 310 of the trend line can be displayed in a first color (e.g., green) to indicate that, at an indicated time, the user's analyte levels are within target analyte range 308. Other portions 312 of the trend line can be displayed in a second color (e.g., orange) to indicate that, at a different indicated time, the user's analyte levels are above target analyte range 308. Similarly, other portions 318 of the trend line can be displayed in a third color (e.g., red) to indicate that, at a different indicated time, the user's analyte levels are below target analyte range 308. In some embodiments, the respective areas under (or above) the trend line can be shaded or filled (e.g., in a particular color) if the user's analyte levels are above or below the target analyte range. For example, the respective areas under (or above) the trend line can be shaded or filled using the second color if the user's analyte levels are above the target analyte range, and/or shaded or filled using the third color if the user's analyte levels are below the target analyte range. As shown in FIG. 3A, the colored areas can extend from the line indicating the exceeded analyte threshold value to the trend line. In this regard, these colored areas can graphically represent to the user the severity and/or duration of an analyte excursion. By contrast, if the user stays within the target analyte range 308 during the predetermined time period, the trend line remains colored according to the first color (e.g., green).

According to some embodiments, the respective areas under (or above) the trend line can remain uncolored. In still other embodiments, the trend line can be a single color regardless of whether the analyte levels are above, below, or within target analyte range 308.

According to some embodiments, certain information can also be superimposed on the graph proximate to the trend line. For example, in certain analyte monitoring systems, user-initiated analyte checks 315 can be graphically represented as one or more discrete points on the graph to indicate the time(s) at which said checks occurred. A user-initiated analyte check can be, for example, a finger stick blood glucose test, a scan of the sensor control device, a download of analyte data from a trusted computer system, a view of the analyte monitoring program on the reader device, and/or a rendering of a specific GUI (e.g., home screen, sensor results screen) of the analyte monitoring program on the reader device. Those of skill in the art will appreciate that other types of user-initiated analyte checks are possible and within the scope of the present disclosure. According to another aspect of some embodiments, exercise events can be graphically represented as an exercise icon 316 on the graph to indicate the time(s) at which said exercise events occurred. The information (e.g., corresponding to events such as user-initiated analyte checks 315 and exercise icon 316) may be displayed on the graph or adjacent to the graph, such as aligned with the x-axis.

According to another aspect of some embodiments, GUI 300 can comprise one or more summary metrics. For example, in some embodiments, GUI 300 can include a “Time in Range” metric 304 to indicate the percentage of time (e.g., “79%”) that a user's analyte data was within a target analyte range for the predetermined time period. In other embodiments, the “Time in Range” metric 304 can be displayed as a graphical representation (e.g., a pie chart, a plurality of bar portions, a partially-filled ring), or, in the alternative, a numeric amount of time. Other types of graphical representations for “Time in Range” metrics are described in U.S. Publ. Nos. 2021/0282673, 2022/0248988, 2021/037860, 2022/0000399, 2021/0030323, 2022/0092019, and 2022/0110551, all of which are incorporated by reference in their entireties for all purposes.

Referring still to FIG. 3A, the summary metrics can also include total amounts of food or medication 306 taken during the predetermined time period. In some embodiments, for example, the summary metrics can include the amount of carbohydrates (in units of grams) that was ingested by the user during the predetermined time period, and/or the type (e.g., rapid-acting or long-acting) and the number of units of insulin injected by the user during the predetermined time period. The summary metrics may include a separate metric for different types of insulin (e.g., rapid-acting and long-acting), or these may be combined. According to another aspect of the embodiments, the specific food and/or medication events 314 can be displayed next to the graph (e.g., above trend line, below trend line), such that each food and/or medication event is aligned with an indicated time reflected along the x-axis. According to one aspect of some embodiments, each type of summary metric can be shown in a different color associated with the associated event(s) shown in a graph section of report GUI 300, as described below.

Referring next to FIG. 3B, an example embodiment of a report GUI 320 for an analyte monitoring system is depicted, in which report GUI 320 includes a plurality of interfaces 320-A, 320-B, 320-C, 320-D, and 320-E, each of which depicts a user's analyte levels over a different predetermined time period in conjunction with a plurality of summary metrics. As an initial matter, each interface of the plurality of interfaces 320-A, 320-B, 320-C, and 320-E includes features similar to those of GUI 300 of FIG. 3A. Accordingly, a detailed description of those similar features will not be repeated here. For example, interface 320-A depicts a daily view of a user's analyte levels for Thursday, May 31, along with a plurality of summary metrics for that particular day. Similarly, interface 320-B depicts a daily view of a user's analyte levels for Friday, June 1, along with a plurality of summary metrics for that particular day. In other words, the report GUI 320 includes a daily view of a user's analyte levels for a plurality of predetermined time periods, for example for a plurality of days. Unlike GUI 300 of FIG. 3A, however, the interfaces of report GUI 320 show labeling for the y-axis and the target analyte range along the right side of each graph, instead of the left side. In other examples, the labeling may be on the left side.

According to another aspect of the embodiments, report GUI 320 can have a specific arrangement that enhances a user's ability to recognize patterns across multiple time periods. For example, the x-axis of each of interfaces 320-A, 320-B, 320-C, 320-D, and 320-E can be aligned with each other such that the specific time increments for each interface are also aligned. In this regard, a user is able to quickly recognize if her analyte levels are exceeding an analyte threshold at a particular time of the day (e.g., after lunch).

According to another aspect of the embodiments, report GUI 320 can include the name of the user 322, a label indicating a selected reporting period 324, and a “Time CGM Active” statistic 325 to indicate the amount of analyte data available during the selected reporting period 324. According to some embodiments, the selected reporting period 324 can be configurable by the user. For example, in some embodiments, the selected reporting period 324 can be configured by a selected start date and a selected end date. In other embodiments, the selected reporting period 324 can be configured to a specific period of time (e.g., one week, two weeks, one month, etc.) Furthermore, in some embodiments, the “Time CGM Active” statistic can comprise a percentage of time during the selected reporting period 324 for which analyte data was obtained. In other embodiments, the “Time CGM Active” statistic can comprise an actual amount of time (e.g., “12 Days, 11 Hours, 32 Minutes”). Report GUI 320 can also include a legend 326 providing textual descriptions of one or more icons that are displayed in any one or more of the interfaces 320-A, 320-B, 320-C, 320-D, and/or 320-E.

According to another aspect of the embodiments, report GUI 320 can also include a device label 328 to indicate the types of devices and/or software used to obtain the analyte data for the report. For example, device label 328 can indicate that a user was using a particular analyte monitoring program on their smart phone in conjunction with a particular brand and/or model of an on-body analyte sensor. In some embodiments, device label 328 can also indicate whether data from a medication delivery device (e.g., a connected insulin pen, insulin pump, etc.) was obtained for report GUI 320.

Referring next to FIG. 3C, another example embodiment of a GUI 330 for an analyte monitoring system is depicted, in which GUI 330 depicts a user's analyte levels over a predetermined time period in conjunction with a plurality of summary metrics. As an initial matter, GUI 330 includes features similar to those of GUI 300 of FIG. 3A. Accordingly, a detailed description of those similar features will not be repeated here.

According to one aspect of the embodiments, the plurality of summary metrics of GUI 330 can include a maximum analyte level 332 and a minimum analyte level 334 for the predetermined time period. In addition, maximum analyte levels 336 and minimum analyte levels 338 for each time increment in a plurality of time increments can be displayed along the x-axis and adjacent to the graph portion of GUI 330. For example, as shown in FIG. 3C, a pair of maximum analyte level 336 and minimum analyte level 338 metrics are displayed for each hour within a twenty-four hour period. The maximum analyte levels 336 and minimum analyte levels 337 can be particularly useful since, in many of the embodiments of the present disclosure, the numeric values associated with the trend line is not always easily ascertainable by visual inspection.

Referring next to FIG. 3D, another example embodiment of a GUI 340 for an analyte monitoring system is depicted, in which GUI 340 depicts a user's analyte levels over a predetermined time period in conjunction with a plurality of summary metrics. As an initial matter, GUI 340 includes features similar to those of GUI 330 of FIG. 3B. Accordingly, a detailed description of those similar features will not be repeated here.

According to one aspect of the embodiments, GUI 340 can include an icon area for the display of certain events along the x-axis. For example, in some embodiments, an alarm icon 342 can be displayed in the icon area to indicate the detection of an alarm condition. Further, the position of alarm icon 342 along the x-axis can indicate to the user the specific time at which the alarm condition was detected. In addition, according to some embodiments, an alarm label 341 can be displayed next to the alarm icon 342 to indicate the type of alarm condition. As seen in FIG. 3D, for example, an alarm icon 342 is displayed at 3:00 am with an alarm label 341 indicating the detection of a “low” analyte alarm condition at that time.

According to some embodiments, other alarm conditions for which an alarm label can be displayed can include a “high” analyte alarm condition, an “urgent low” or “serious low” analyte alarm condition, a “projected high” analyte alarm condition, a “projected low” analyte alarm condition, a “falling fast” analyte alarm condition, a “rising fast” analyte alarm condition, a “signal loss” condition, among others.

According to another aspect of the embodiments, a carbohydrate icon 343 and/or a medication icon 344 can be displayed in the icon area to indicate, respectively, a meal event or an insulin event. In addition, according to some embodiments, the specific amount of carbohydrates (in units of grams) and/or the type (e.g., rapid-acting or long-acting) and the number of units of insulin injected by the user can also be displayed next to the carbohydrate icon 343 or medication icon 344. Accordingly, a user will be able to visually ascertain, at a glance, whether certain events (e.g., meal or insulin), and specific details regarding those events (e.g., grams of carbohydrates or units of rapid-acting insulin), has had an effect on the trend line.

In addition, although the icons are shown in an icon area that is separate from the graph portion of GUI 340, those of skill in the art will appreciate that, in alternative embodiments, the icons can be displayed directly on the graph adjacent to the trend line. Likewise, details of the alarm condition, grams of carbohydrates, and/or units of insulin can also be displayed directly on the graph adjacent to the trend line. In some examples, information corresponding to events, such as, for example, user-initiated analyte checks 315 and/or exercise icons 316 may be displayed in the icon area along the x-axis (for example, instead of on the graph portion adjacent to the trend line).

Referring next to FIG. 3E, another example embodiment of a report GUI 345 for an analyte monitoring system is depicted, in which report GUI 345 includes a plurality of interfaces 345-A, 345-B, and 345-C, each of which depicts a user's analyte levels over a different predetermined time period in conjunction with a plurality of summary metrics. As an initial matter, report GUI 345 includes features similar to those of report GUI 320 of FIG. 3B. Additionally, each of the plurality of interfaces 345-A, 345-B, and 345-C includes features similar to those of GUI 340 of FIG. 3D. Accordingly, a detailed description of those similar features will not be repeated here.

Referring next to FIG. 3F, another example embodiment of a report GUI 350 for an analyte monitoring system is depicted, in which report GUI 350 includes a plurality of interfaces 350-A, 350-B, and 350-C, each of which depicts a user's analyte levels over a different predetermined time period in conjunction with a plurality of summary metrics. As an initial matter, report GUI 350 includes features similar to those of report GUI 345 of FIG. 3E, except that report GUI 350 does not include alarm labels. Accordingly, a detailed description of those similar features will not be repeated here.

Referring next to FIG. 3G, another example embodiment of a GUI 355 for an analyte monitoring system is depicted, in which GUI 335 depicts a user's analyte levels over a predetermined time period in conjunction with a plurality of summary metrics. As an initial matter, GUI 355 includes features similar to those of GUI 330 of FIG. 3C. Accordingly, a detailed description of those similar features will not be repeated here.

According to one aspect of the embodiments, GUI 355 can include a maximum analyte level 356 and a minimum analyte level 357 for the predetermined time period. Likewise, maximum analyte levels 359 and minimum analyte levels 358 for each time increment (e.g., every hour) can be displayed along the x-axis and adjacent to the graph portion. In addition, as can be seen in FIG. 3G, if either of the minimum or the maximum analyte levels are above the target analyte range, then the numeric value can be displayed in a first color (e.g., orange). The first color may correspond to the color of portions of the trend line and/or the shaded area if the user's analyte levels are above the target analyte range. Similarly, if either of the minimum or the maximum analyte levels are below the target analyte range, then the numeric value can be displayed in a second color (e.g., red). The second color may correspond to the color of the portions of the trend line and/or the shaded area if the user's analyte levels are below the target analyte range. By contrast, if either of the minimum or the maximum analyte levels are within the target analyte range, then the numeric value can be displayed without any coloring. Alternatively, the numeric value can be displayed in a color corresponding to the color of the portions of the trend line if the user's analyte levels are within the target analyte range. Accordingly, GUI 355 draws the user's attention to those portions of the trend line, and the corresponding minimum or maximum analyte level numeric values, that are outside the target analyte range to allow the user to ascertain the underlying reasons.

Referring next to FIG. 3H, another example embodiment of a GUI 360 for an analyte monitoring system is depicted, in which GUI 360 depicts a user's analyte levels over a predetermined time period in conjunction with a plurality of summary metrics. As an initial matter, GUI 360 includes features similar to those of GUI 355 of FIG. 3G, except for the color scheme. In particular, if either of a minimum or maximum analyte level is above the target analyte range, then the numeric value is displayed in a first muted color (e.g., yellow). The muted color may correspond to a muted shade of the color of the portions of the trend line and/or the shaded area if the user's analyte levels are above the target analyte range. A detailed description of the similar features will not be repeated here.

Referring next to FIG. 3I, another example embodiment of a report GUI 365 for an analyte monitoring system is depicted, in which report GUI 365 includes a plurality of interfaces 365-A, 365-B, 365-C, and 365-D, each of which depicts a user's analyte levels over a different predetermined time period in conjunction with a plurality of summary metrics. As an initial matter, report GUI 365 includes features similar to those of report GUI 350 of FIG. 3F. Additionally, each of the plurality of interfaces, 365-A, 365-B, 365-C, and 365-D, includes features similar to those of GUI 360 of FIG. 3H. Accordingly, a detailed description of those similar features will not be repeated here.

Referring next to FIG. 3J, another example embodiment of a GUI 370 for an analyte monitoring system is depicted, in which GUI 370 depicts a user's analyte levels over a predetermined time period in conjunction with a plurality of summary metrics. As an initial matter, GUI 370 includes features similar to those of GUI 355 of FIG. 3G. For example, GUI 370 includes maximum analyte levels 372 and minimum analyte levels 374 for each time increment (e.g., every hour) which are displayed along the x-axis and adjacent to the graph portion. In addition, as can be seen in FIG. 3J, if either of the minimum or the maximum analyte levels are above the target analyte range, then the numeric value can be displayed in a first color (e.g., orange). Similarly, if either of the minimum or the maximum analyte levels are below the target analyte range, then the numeric value can be displayed in a second color (e.g., red). By contrast, if either of the minimum or the maximum analyte levels are within the target analyte range, then the numeric value can be displayed without any coloring.

According to some embodiments, however, GUI 370 can have numerous differences from GUI 355 of FIG. 3G. In particular, GUI 370 does not include some of the summary metrics present in several of the previously described embodiments. For example, GUI 370 does not include a “Time in Range” metric. In addition, GUI 370 does not include a maximum analyte level or a minimum analyte level for the entire predetermined time period. Similarly, GUI 370 does not include the total amounts of food or medication taken during the predetermined time period. GUI 370 can be configured to not display any medication information, and to only include meal information. For example, in some embodiments, GUI 370 can include a carbohydrate icon 376 displayed directly on the graph portion, along with an amount of the carbohydrates 378 displayed next (e.g., below) the graph. In other examples, GUI 370 can be configured to not display any meal information, and to only include medication information.

Referring next to FIG. 3K, another example embodiment of a report GUI 380 for an analyte monitoring system is depicted, in which report GUI 380 includes a plurality of interfaces 380-A, 380-B, 380-C, and 380-D, each of which depicts a user's analyte levels over a different predetermined time period in conjunction with a plurality of summary metrics. As an initial matter, report GUI 380 includes features similar to those of report GUI 365 of FIG. 3I. Additionally, each of the plurality of interfaces, 380-A, 380-B, 380-C, and 380-D, includes features similar to those of GUI 360 of FIG. 3H. Accordingly, a detailed description of those similar features will not be repeated here.

According to one aspect of the embodiments, however, GUI 380 differs from previous embodiments in that each of the interfaces 380-A, 380-B, 380-C, and 380-D will only display minimum analyte levels 382 or maximum analyte levels 384 for each time increment (e.g., every hour) if they are outside the target analyte range. For example, if a minimum analyte level 382 is below the target analyte range, then the numeric value can be displayed in a first color (e.g., red). Similarly, if a maximum analyte level 384 is above the target analyte range, then the numeric value can be displayed in a second color (e.g., yellow). By contrast, however, if a minimum analyte level or maximum analyte level for a particular time increment is within the target analyte range, then no numeric value is displayed to the user. In this manner, GUI 380 can highlight numeric values that fall outside the target analyte range, allowing the user to better focus on ascertaining the underlying causes of such analyte excursions. In other words, the minimum and maximum analyte levels may be displayed if they are above or below the target analyte range, but not if they are within the target analyte range.

Referring next to FIG. 3L, another example embodiment of a report GUI 385 for an analyte monitoring system is depicted, in which report GUI 385 includes a plurality of interfaces 385-A, 385-B, and 385-C, each of which depicts a user's analyte levels over a different predetermined time period in conjunction with a plurality of summary metrics. As an initial matter, report GUI 385 includes features similar to those of report GUI 350 of FIG. 3F. Additionally, each of the plurality of interfaces, 385-A, 385-B, and 385-C, includes features similar to those of GUI 330 of FIG. 3C. Accordingly, a detailed description of those similar features will not be repeated here.

According to one aspect of the embodiments, however, GUI 385 differs from previous embodiments in that interfaces 385-A, 385-B, and 385-C can each also include a graphical representation of an amount of the medication taken. For example, interface 385-C depicts a first bar portion 387 sized proportionally to the amount of rapid-acting insulin taken around 7:00 am, which in this case is “1u.” In addition, interface 385-C depicts a second bar portion 388 sized proportionally to the amount of rapid-acting insulin taken around 11:00 am, which in this case is “3u.” Accordingly, the second bar portion 388 is larger than the first bar portion 387. Furthermore, interface 385-C depicts a medication icon 389 that is labeled with “20 u” to indicate a dose of long-acting insulin taken around 9:00 pm. According to some embodiments, icon 389 is used to reflect a different type of insulin (e.g., rapid-acting versus long-acting) and/or to account for scale (i.e., the bar portion for 20u would not fit on the chart if scaled according to the first and the second bar portions).

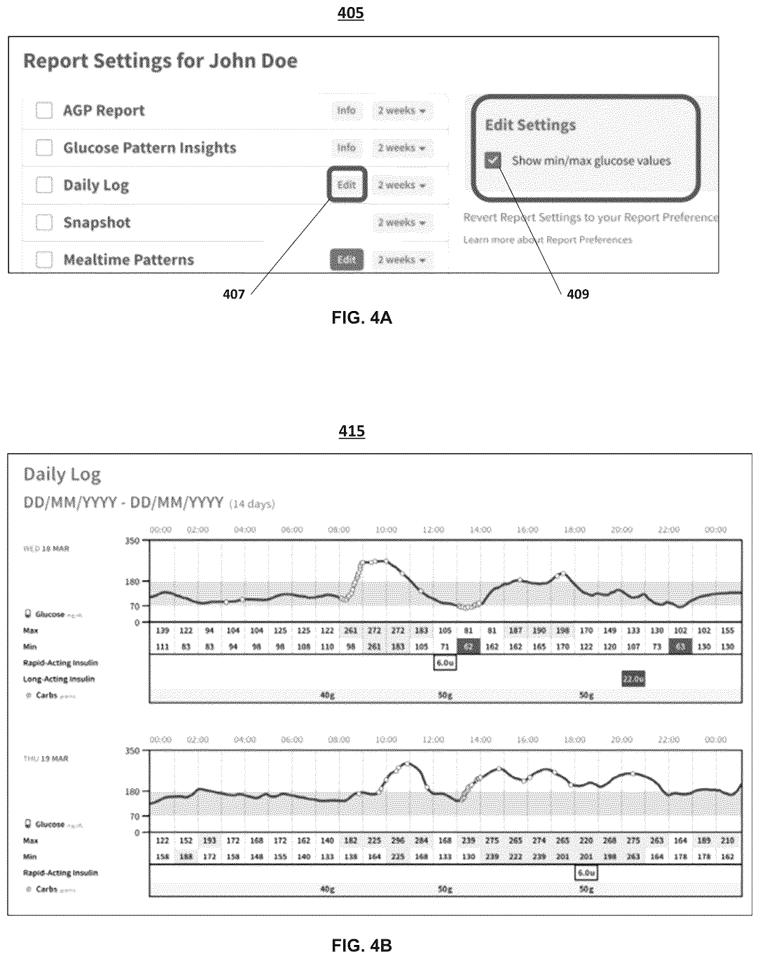

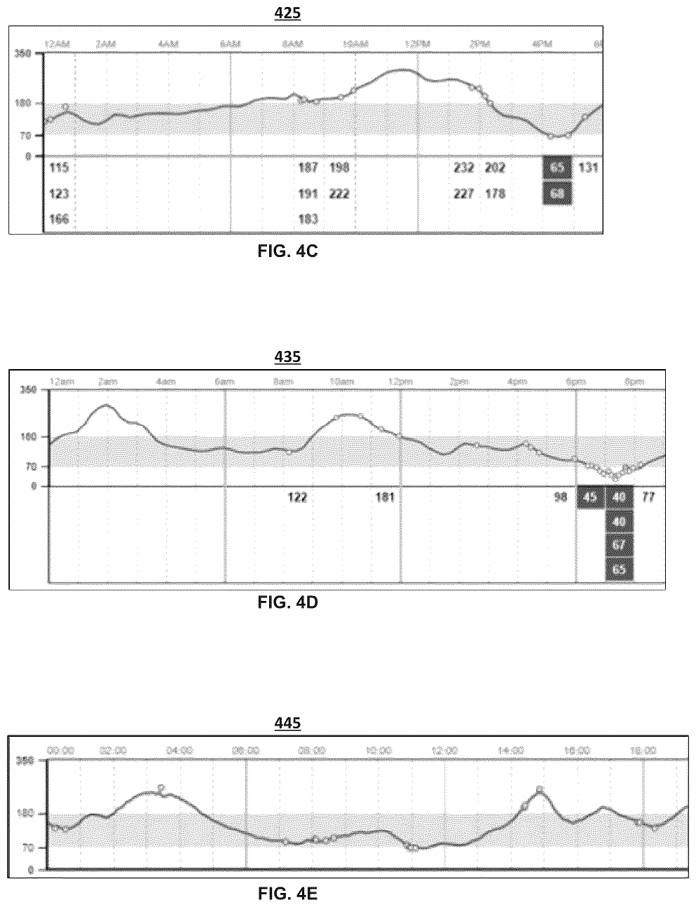

Referring next to FIG. 4A, an example embodiment of a report settings GUI 405 for an analyte monitoring system is depicted. According to one aspect of some embodiments, the display of minimum analyte levels and maximum analyte levels can be enabled and/or disabled through a report settings GUI 405. In some embodiments, for example, report settings GUI 405 can include an edit button 407 which, when pressed, further presents a checkbox 409 that allows the user to enable or disable the display of minimum analyte levels and maximum analyte levels. In some embodiments, checkbox 409 can be presented via a separate interface, such as, for example, a modal. In other embodiments, checkbox 409 can be presented on the report settings GUI 405. Further, although checkbox 409 is shown in the depicted embodiments, those of skill in the art will appreciate that other GUI objects and/or features can be implemented, such as, for example, radio buttons, sliders, dropdown menus, switches, and the like. In some embodiments, the display of minimum analyte levels and maximum analyte levels can be enabled by default. In other embodiments, the display of minimum analyte levels and maximum analyte levels can be disabled by default.

Referring next to FIG. 4B, an example embodiment of a report GUI 415 for an analyte monitoring system is depicted, where the display of minimum analyte levels and maximum analyte levels have been enabled via report settings GUI 405 (FIG. 4A). In many respects, report GUI 415 includes features similar to those of GUI 360 (FIG. 3H) and report GUI 365 (FIG. 3I). As such, a detailed description of similar features will not be repeated here. It should also be noted that the functionality to enable and/or disable the display of minimum analyte levels and maximum analyte levels can be implemented with any of the GUIs and report GUIs described herein that include minimum analyte levels and maximum analyte levels.

Referring next to FIGS. 4C to 4E, example embodiments of GUIs for an analyte monitoring system are depicted. According to one aspect of the embodiments, GUIs 425, 235, and 445 are example embodiments of GUIs configured to display a user's analyte levels over a predetermined time period, and where the display of minimum analyte levels and maximum analyte levels have been disabled via report settings GUI 405 (FIG. 4A). For example, FIG. 4C is an example embodiment of a GUI 425 depicting a user's analyte levels over a predetermined time period, where analyte data is transmitted from a sensor control device to a reader device in response to a scan or request for data by the reader device (e.g., via NFC). As another example, FIG. 4D is an example embodiment of a GUI 435 depicting a user's analyte levels over a predetermined time period, where analyte data can be transmitted both automatically (e.g., via Bluetooth) and in response to a scan or request for data by the reader device (e.g., via NFC). As yet another example, FIG. 4E is an example embodiment of a GUI 445 depicting a user's analyte levels over a predetermined time period, where analyte data is transmitted automatically from a sensor control device to a reader device (e.g., via Bluetooth). Furthermore, although not shown, according to many embodiments, GUIs 425, 435, and 445 can also be configured to display a graph portion in conjunction with a plurality of summary metrics, such as those shown in FIGS. 3A, 3B, and elsewhere throughout the present disclosure.

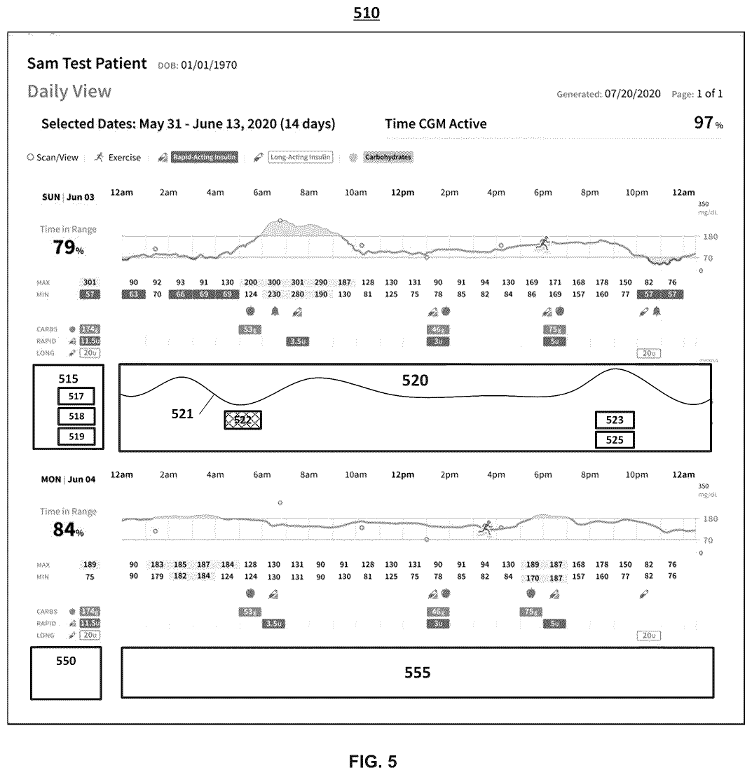

According to another aspect of some embodiments, information relating to multiple analytes can be displayed on a GUI or GUI report in a similar manner to the previously disclosed embodiments. FIG. 5 is an example embodiment of a report GUI 510 for an analyte monitoring system, in which report GUI 510 includes a plurality of interfaces, each of which depicts a user's glucose levels over a different predetermined time period in conjunction with a plurality of summary glucose metrics. In many respects, GUI 510 includes features similar to those in the report GUI described with respect to FIG. 3I. Accordingly, a detailed description of those similar features will not be repeated here.

According to another aspect of some embodiments, report GUI 510 can further include information relating to a second analyte, such as, for example, ketone. In particular, a plurality of interfaces is provided, each of which depicts a user's glucose levels and ketone levels over a predetermined time period. In addition, in some embodiments, summary metrics 515 can be displayed for a second analyte (e.g., ketones), including, for example, a number of high ketone threshold alarms 517, a number of elevated ketone threshold alarms 518, and/or a maximum ketone level 519 for the predetermined time period. Furthermore, in some embodiments, a graph portion 520, adjacent to the glucose graph portion, can depict ketone levels over the predetermined time period, such that graph portion 520 is aligned along the same x-axis (e.g., time) as the glucose graph portion. As shown in FIG. 5, in some embodiments, graph portion 520 can comprise a trend line 521 that depicts the user's ketone levels over the predetermined time period. In other embodiments, discrete points of data can be displayed instead of trend line 521. Additionally, graph portion 520 can also include numeric values 522, 523 that exceed a high ketone threshold or an elevated ketone threshold. As seen in FIG. 5, the numeric values can be displayed with a distinct shading or coloring associated with the particular threshold. In some embodiments, graph portion 520 can also include an alarm indicator 525 (e.g., text, marker, icon, or symbol) to indicate to the user when a ketone alarm condition has been triggered. According to an aspect of some embodiments, the numeric values 522, 523 and alarm indicator 525 can be positioned on graph portion 520 such that they are aligned on the same x-axis (e.g., time) as trend line 521 and the glucose graph portion.

According to another aspect of some embodiments, report GUI 510 can be configured such that if the levels of the second analyte (e.g., ketone) are within a predetermined target range during a predetermined time period, then a trend line will not be displayed. Referring again to FIG. 5, an example embodiment of a second interface comprising a second summary metrics portion 550 and a second graph portion 555 are shown below the first interface described earlier. Here, the levels of the second analyte are within a predetermined target range during the second predetermined time period (e.g., “MON, June 4”). Accordingly, second summary metrics 550 can indicate that no alarms were activated during the second predetermined time period. Furthermore, second graph portion 555 does not include a trend line, but instead can include an indicator (e.g., a text, marker, icon, or symbol) that the levels for the second analyte were “normal” during the second predetermined time period. In some embodiments, second graph portion 555 can be more compact relative to first graph portion 520, since less area of report GUI 510 is needed.

It will be understood by those of skill in the art that any of the GUIs, reports interfaces, or portions thereof, as described herein, are meant to be illustrative only, and that the individual elements, or any combination of elements, depicted and/or described for a particular embodiment or figure are freely combinable with any elements, or any combination of elements, depicted and/or described with respect to any of the other embodiments. For example, the display of meal information (e.g., amount of carbohydrate) and medication information (e.g., rapid-lasting and long-lasting insulin) for each time increment may be provided or omitted in each of the embodiments described. In addition, the display of alarm icons and alarm labels for the predetermined time period may be provided or omitted in each of the embodiments described. The display of a graphical representation of the amount of medication also may be provided or omitted in each of the embodiments described. The display of summary metrics for the total amounts of meal information and/or medication information over the predetermined time period may be provided or omitted in each of the embodiments described. The “Time in Range” metric may be provided or omitted in each of the embodiments described. The coloring of the graph (e.g., the trend line, the threshold lines, and/or the shaded portions) may be provided or omitted in each of the embodiments described.

In some embodiments, data indicative of the user's analyte levels can be wirelessly transmitted from the sensor control device 102 to the reader device 120, for example, automatically (i.e., without user intervention). For example, the sensor control device 102 may periodically transmit analyte data to the reader device (e.g., by Bluetooth or BLE) without requiring the user to manually perform a scan (e.g., by NFC).

In analyte monitoring systems where data indicative of a user's analyte levels is wirelessly and automatically transmitted from a sensor control device 102 to a reader device 120, the embodiments described herein can be advantageous in that numeric values associated with a subject's analyte levels for each time increment with a predetermined time period (or a subset thereof) can be regularly displayed, such that the user (or her HCP) can make more informed treatment decisions. The numeric values associated with each time increment can be further correlated with events—such as medication, exercise, and/or meals—thereby providing the user (or her HCP) with a robust overview of the user's overall glycemic control over a predetermined time period.

It will also be understood by those of skill in the art that any of the GUIs, reports interfaces, or portions thereof, as described herein, can be implemented in an analyte monitoring system for monitoring one or more types of analytes. These analytes can include one or more of glucose, ketones, lactate, alcohol, or any other analytes that are detectable in a bodily fluid of the user. Preferably, the analyte comprises glucose. It will also be understood by those of skill in the art that the GUIs, reports interfaces, or portions thereof, as described herein, can incorporate data received from multiple analyte monitoring systems and devices relating thereto, including the incorporation of data from devices for taking ex vivo analyte measurements and/or physiological measurements.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

In summary, improved graphical user interfaces for analyte monitoring systems are provided. For example, disclosed herein are various embodiments of GUIs, each of which include a graph portion comprising a trend line indicative of a user's analyte levels over a predetermined time period, and a plurality of summary metrics comprising a plurality of minimum analyte levels and maximum analyte levels associated with a plurality of time increments within the predetermined time period. In many embodiments, the plurality of minimum analyte levels and maximum analyte levels are aligned with an x-axis of the graph portion.

The disclosure of this application also contains the following numbered clauses:
1. An analyte monitoring system, comprising:
   a sensor control device comprising an analyte sensor coupled with sensor electronics, the sensor control device configured to be worn on a user's body and transmit data indicative of the user's analyte levels; and
   a reader device comprising:
      a display;
      wireless communication circuitry configured to receive the data indicative of the user's analyte levels;
      one or more processors coupled with a memory, the memory configured to store instructions that, when executed by the one or more processors, cause the one or more processors to output to the display a graphical user interface (GUI) comprising:
         a graph portion comprising a trend line indicative of the user's analyte levels over a predetermined time period, wherein the trend line is based on the received data indicative of the user's analyte levels; and
         a plurality of summary metrics associated with the predetermined time period, wherein the plurality of summary metrics comprises a plurality of minimum analyte levels and maximum analyte levels associated with a plurality of time increments within the predetermined time period, and
   wherein the graph portion includes an x-axis comprising units of time, and wherein the plurality of minimum analyte levels and maximum analyte levels are aligned with the x-axis of the graph portion.
2. The analyte monitoring system of clause 1, wherein the predetermined time period is one day.
3. The analyte monitoring system of clause 1 or 2, wherein the plurality of time increments comprises twenty-four one-hour increments.
4. The analyte monitoring system of any preceding clause, wherein the x-axis comprises a plurality of labels arranged in two-hour increments.
5. The analyte monitoring system of any preceding clause, wherein the graph portion further includes a y-axis comprising units indicative of analyte level concentrations.
6. The analyte monitoring system of any preceding clause, wherein the graph portion further comprises a first label for a high analyte threshold value and a second label for a low analyte threshold value.
7. The analyte monitoring system of any preceding clause, wherein the graph portion further defines a first line indicative of a high analyte threshold value and a second line indicative of a low analyte threshold value.
8. The analyte monitoring system of any preceding clause, wherein the plurality of minimum analyte levels and maximum analyte levels comprises:
   one or more minimum analyte levels depicted in a first color if the one or more minimum analyte levels are above a high analyte level threshold value; and
   one or more maximum analyte levels depicted in the first color if the one or more maximum analyte levels are above the high analyte level threshold value.
9. The analyte monitoring system of clause 8, wherein the plurality of minimum analyte levels and maximum analyte levels further comprises:
   one or more minimum analyte levels depicted in a second color if the one or more minimum analyte levels are below a low analyte level threshold value; and
   one or more maximum analyte levels depicted in the second color if the one or more maximum analyte levels are below the low analyte level threshold value.
10. The analyte monitoring system of any preceding clause, wherein the plurality of summary metrics further comprises a time in range metric.
11. The analyte monitoring system of clause 10, wherein the time in range metric is displayed as a percentage.
12. The analyte monitoring system of any preceding clause, wherein the plurality of summary metrics further comprises a total amount of carbohydrates consumed during the predetermined time period.
13. The analyte monitoring system of any preceding clause, wherein the plurality of summary metrics further comprises a total amount of medication taken during the predetermined time period.
14. The analyte monitoring system of any preceding clause, wherein the trend line comprises a first portion in a first color and a second portion in a second color, wherein the first portion is indicative of analyte levels above a high analyte threshold value, and wherein the second portion is indicative of analyte levels between a low analyte threshold value and the high analyte threshold value.
15. The analyte monitoring system of clause 14, wherein the trend line further comprises a third portion in a third color, wherein the third portion is indicative of analyte levels below the low analyte threshold value.
16. The analyte monitoring system of any preceding clause, further comprising an icon area configured to display a plurality of event icons, wherein the plurality of event icons includes at least one of: an alarm icon, a carbohydrate icon, and a medication icon.
17. The analyte monitoring system of clause 16, wherein the alarm icon comprises an alarm label configured to textually describe an alarm condition associated with the alarm icon.
18. The analyte monitoring system of clause 16 or 17, wherein the alarm condition is selected from a group of a high analyte alarm condition, an urgent low analyte alarm condition, a projected high analyte alarm condition, a projected low analyte alarm condition, a falling fast analyte alarm condition, a rising fast analyte alarm condition, and a signal loss condition.
19. The analyte monitoring system of any of clauses 16 to 18, wherein the plurality of event icons is configured to be displayed in alignment with the x-axis of the graph portion.
20. The analyte monitoring system of any preceding clause, wherein the data indicative of the user's analyte levels comprises glucose data.

What is claimed is:

1. An analyte monitoring system, comprising:
a sensor control device comprising an analyte sensor coupled with sensor electronics, the sensor control device configured to be worn on a user's body and transmit data indicative of the user's analyte levels; and
a reader device comprising:
a display;
wireless communication circuitry configured to receive the data indicative of the user's analyte levels;
one or more processors coupled with a memory, the memory configured to store instructions that, when executed by the one or more processors, cause the one or more processors to output to the display a graphical user interface (GUI) comprising:
a graph portion comprising a trend portion indicative of the user's analyte levels over a predetermined time period, wherein the trend portion is based on the received data indicative of the user's analyte levels; and
a plurality of summary metrics associated with the predetermined time period, wherein the plurality of summary metrics comprises a minimum analyte level for the predetermined time period and a maximum analyte level for the predetermined time period, and
wherein the graph portion further comprises an x-axis comprising units of time, and wherein a plurality of minimum analyte levels for a plurality of time increments and a plurality of maximum analyte levels for the plurality of time increments are aligned with the x-axis of the graph portion.

2. The analyte monitoring system of claim 1, wherein the predetermined time period is one day.

3. The analyte monitoring system of claim 1, wherein the plurality of time increments comprises twenty-four one-hour increments.

4. The analyte monitoring system of claim 3, wherein the x-axis comprises a plurality of labels arranged in two-hour increments.

5. The analyte monitoring system of claim 1, wherein the graph portion further includes a y-axis comprising units indicative of analyte level concentrations.

6. The analyte monitoring system of claim 1, wherein the graph portion further comprises a first label for a high analyte threshold value and a second label for a low analyte threshold value.

7. The analyte monitoring system of claim 6, wherein the graph portion further defines a first line indicative of the high analyte threshold value and a second line indicative of the low analyte threshold value.

8. The analyte monitoring system of claim 6, wherein the plurality of minimum analyte levels for the plurality of time increments and the plurality of maximum analyte levels for the plurality of time increments comprise:
one or more minimum analyte levels depicted in a first color if the one or more minimum analyte levels are above the high analyte level threshold value; and
one or more maximum analyte levels depicted in the first color if the one or more maximum analyte levels are above the high analyte level threshold value.

9. The analyte monitoring system of claim 8, wherein the plurality of minimum analyte levels for the plurality of time increments and the plurality of maximum analyte levels for the plurality of time increments further comprise:
one or more minimum analyte levels depicted in a second color if the one or more minimum analyte levels are below the low analyte level threshold value; and
one or more maximum analyte levels depicted in the second color if the one or more maximum analyte levels are below the low analyte level threshold value.

10. The analyte monitoring system of claim 1, wherein the plurality of summary metrics further comprises a time in range metric.

11. The analyte monitoring system of claim 10, wherein the time in range metric is displayed as a percentage.

12. The analyte monitoring system of claim 1, wherein the plurality of summary metrics further comprises a total amount of carbohydrates consumed during the predetermined time period.

13. The analyte monitoring system of claim 1, wherein the plurality of summary metrics further comprises a total amount of medication taken during the predetermined time period.

14. The analyte monitoring system of claim 6, wherein the trend portion comprises a first portion in a first color and a second portion in a second color, wherein the first portion is indicative of analyte levels above the high analyte threshold value, and wherein the second portion is indicative of analyte levels between the low analyte threshold value and the high analyte threshold value.

15. The analyte monitoring system of claim 14, wherein the trend portion further comprises a third portion in a third color, wherein the third portion is indicative of analyte levels below the low analyte threshold value.

16. The analyte monitoring system of claim 1, further comprising an icon area configured to display a plurality of event icons, wherein the plurality of event icons includes at least one of: an alarm icon, a carbohydrate icon, and a medication icon.

17. The analyte monitoring system of claim 16, wherein the alarm icon comprises an alarm label configured to textually describe an alarm condition associated with the alarm icon.

18. The analyte monitoring system of claim 16, wherein the alarm condition is selected from a group of a high analyte alarm condition, an urgent low analyte alarm condition, a projected high analyte alarm condition, a projected low analyte alarm condition, a falling fast analyte alarm condition, a rising fast analyte alarm condition, and a signal loss condition.

19. The analyte monitoring system of claim 16, wherein the plurality of event icons is configured to be displayed in alignment with the x-axis of the graph portion.

20. The analyte monitoring system of claim 1, wherein the data indicative of the user's analyte levels comprises glucose data.

21. The analyte monitoring system of claim 1, wherein the trend portion is a continuous trend line.

22. The analyte monitoring system of claim 1, wherein the trend portion comprises a plurality of discrete data points.
